SEQUENCE LISTING (22 sequences)

SEQ ID NO: 1 - protein, 361 aa, Homo sapiens
MAGGRHRRVVGTLHLLLLVAALPWASRGVSPSASAWPEEKNYHQPAILNSSALRQIAEGTSISEMWQNDLQPLLIERYPGSPGSYAARQHIMQRIQRLQADWVLEIDTFLSQTPYGYRSFSNIISTLNPTAKRHLVLACHYDSKYFSHWNNRVFVGATDSAVPCAMMLELARALDKKLLSLKTVSDSKPDLSLQLIFFDGEEAFLHWSPQDSLYGSRHLAAKMASTPHPPGARGTSQLHGMDLLVLLDLIGAPNPTFPNFFPNSARWFERLQAIEHELHELGLLKDHSLEGRYFQNYSYGGVIQDDHIPFLRRGVPVLHLIPSPFPEVWHTMDDNEENLDESTIDNLNKILQVFVLEYLHL

SEQ ID NO: 2 - protein, 382 aa, Homo sapiens
MRSGGRGRPRLRLGERGLMEPLLPPKRRLLPRVRLLPLLLALAVGSAFYTIWSGWHRRTEELPLGRELRVPLIGSLPEARLRRVVGQLDPQRLWSTYLRPLLVVRTPGSPGNLQVRKFLEATLRSLTAGWHVELDPFTASTPLGPVDFGNVVATLDPRAARHLTLACHYDSKLFPPGSTPFVGATDSAVPCALLLELAQALDLELSRAKKQAAPVTLQLLFLDGEEALKEWGPKDSLYGSRHLAQLMESIPHSPGPTRIQAIELFMLLDLLGAPNPTFYSHFPRTVRWFHRLRSIEKRLHRLNLLQSHPQEVMYFQPGEPFGSVEDDHIPFLRRGVPVLHLISTPFPAVWHTPADTEVNLHPPTVHNLCRILAVFLAEYLGL

SEQ ID NO: 3 - protein, 364 aa, Homo sapiens [sequence omitted]

SEQ ID NO: 4 - protein, 481 aa, Homo sapiens [sequence omitted]

SEQ ID NO: 5 - protein, 359 aa, Homo sapiens
AAMRSGGRGRPRLRLGERGLMEPLLPPKRRLLPRVRLLPLLLALAVGSAFYTIWSGWHRRTEELPLGRELRVPLIGSLPEARLRRVVGQLDPQRLWSTYLRPLLVVRTPGSPGNLQVRKAAPVTLQLLFLDGEEALKEWGPKDSLYGSRHLAQLMESIPHSPGPTRIQAIELFMLLDLLGAPNPTFYSHFPRTVRWFHRLRSIEKRLHRLNLLQSHPQEVMYFQPGEPFGSVEDDHIPFLRRGVPVLHLISTPFPAVWHTPADTEVNLHPPTVHNLCRILAVFLAEYLGLRAWPMTVERTVREKVPAGASEAQAGSAGVLVCPFHTFVSLCYNWKTFFLLIVSSCHPSRTGKRPLWDDS

SEQ ID NO: 6 - protein, 42 aa, Homo sapiens [sequence omitted]

SEQ ID NO: 7 - protein, 40 aa, Homo sapiens
DAEFRHDSGYEVHHQKLVFFAEDVGSNKGAIIGLMVGGVV

SEQ ID NO: 8 - protein, 40 aa, Homo sapiens
EFRHDSGYEVHHQKLVFFAEDVGSNKGAIIGLMVGGVVIA

SEQ ID NO: 9 - protein, 38 aa, Homo sapiens
EFRHDSGYEVHHQKLVFFAEDVGSNKGAIIGLMVGGVV

SEQ ID NO: 10 - protein, 38 aa, Homo sapiens
DAEFRHDSGYEVHHQKLVFFAEDVGSNKGAIIGLMVGG

SEQ ID NO: 11 - protein, 36 aa, Homo sapiens
EFRHDSGYEVHHQKLVFFAEDVGSNKGAIIGLMVGG

SEQ ID NO: 12 - protein, 34 aa, Homo sapiens
EASNCFAIRHFENKFAVETLICSRTVKKNIIEEN

SEQ ID NO: 13 - protein, 34 aa, Homo sapiens
EASNCFAIRHFENKFAVETLICFNLFLNSQEKHY

SEQ ID NO: 14 - protein, 76 aa, Homo sapiens
QPDAINAPVTCCYNFTNRKISVQRLASYRRITSSKCPKEAVIFKTIVAKEICADPKQKWVQDSMDHLDKQTQTPKT

SEQ ID NO: 15 - protein, 76 aa, Homo sapiens
QPVGINTSTTCCYRFINKKIPKQRLESYRRTTSSHCPREAVIFKTKLDKEICADPTQKWVQDFMKHLDKKTQTPKL

SEQ ID NO: 16 - protein, 76 aa, Homo sapiens
QPDSVSIPITCCFNVINRKIPIQRLESYTRITNIQCPKEAVIFKTKRGKEVCADPKERWVRDSMKHLDQIFQNLKP

SEQ ID NO: 17 - protein, 101 aa, Mus musculus
QITHATETKEVQSSLKAQQGLEIEMFHMGFQDSSDCCLSYNSRIQCSRFIGYFPTSGGCTRPGIIFISKRGFQVCANPSDRRVQRCIERLEQNSQPRTYKQ

SEQ ID NO: 18 - protein, 75 aa, Homo sapiens
QPDALNVPSTCCFTFSSKKISLQRLKSYVITTSRCPQKAVIFRTKLGKEICADPKEKWVQNYMKHLGRKAHTLKT

SEQ ID NO: 19 - protein, 92 aa, Homo sapiens
QFINDAETELMMSKLPLENPVVLNSFHFAADCCTSYISQSIPCSLMKSYFETSSECSKPGVIFLTKKGRQVCAKPSGPGVQDCMKKLKPYSI

SEQ ID NO: 20 - protein, 97 aa, Homo sapiens
QPKVPEWVNTPSTCCLKYYEKVLPRRLVVGYRKALNCHLPAIIFVTKRNREVCTNPNDDWVQEYIKDPNLPLLPTRNLSTVKIITAKNGQPQLLNSQ

SEQ ID NO: 21 - protein, 373 aa, Homo sapiens
QHHGVTKCNITCSKMTSKIPVALLIHYQQNQASCGKRAIILETRQHRLFCADPKEQWVKDAMQHLDRQAAALTRNGGTFEKQIGEVKPRTTPAAGGMDESVVLEPEATGESSSLEPTPSSQEAQRALGTSPELPTGVTGSSGTRLPPTPKAQDGGPVGTELFRVPPVSTAATWQSSAPHQPGPSLWAEAKTSEAPSTQDPSTQASTASSPAPEENAPSEGQRVWGQGQSPRPENSLEREEMGPVPAHTDAFQDWGPGSMAHVSVVPVSSEGTPSREPVASGSWTPKAEEPIHATMDPQRLGVLITPVPDAQAATRRQAVGLLAFLGLLFCLGVAMFTYQSLQGCPRKMAGEMAEGLRYIPRSCGSNSYVLVPV

SEQ ID NO: 22 - protein, 127 aa, Homo sapiens
QGVFEDCCLAYHYPIGWAVLRRAWTYRIQEVSGSCNLPAAIFYLPKRHRKVCGNPKSREVQRAMKLLDARNKVFAKLHHNTQTFQAGPHAVKKLSSGNSKLSSSKFSNPISSSKRNVSLLISANSGL

(19) United States
(12) Patent Application Publication
(10) Pub. No.: US 2010/0028918 A1
(43) Pub. Date: Feb. 4, 2010

(54) GLUTAMINYL CYCLASE AS A DIAGNOSTIC/PROGNOSTIC INDICATOR FOR NEURODEGENERATIVE DISEASES

(75) Inventors: Hans-Ulrich Demuth, Halle/Saale (DE); Stephan Schilling, Halle/Saale (DE); Martin Kleinschmidt, Halle/Saale (DE); Jens-Ulrich Rahfeld, Lieskau (DE); Astrid Kehlen, Halle/Saale (DE); Monique Haegele, Leipzig (DE)

Correspondence Address: SONNENSCHEIN NATH & ROSENTHAL LLP, P.O. BOX 061080, WACKER DRIVE STATION, WILLIS TOWER, CHICAGO, IL, 60606-1080, US

(73) Assignee: PROBIODRUG AG, Halle/Saale (DE)

(21) Appl. No.: 12/533,099

(22) Filed: Jul. 31, 2009

Related U.S. Application Data
(60) Provisional application No. 61/085,154, filed on Jul. 31, 2008.

(51) Int. Cl. G01N 33/53; C12Q 1/00; C12Q 1/48; C07K 16/00

(52) U.S. Cl. 435/7.92; 435/4; 435/15; 530/389.1

(57) ABSTRACT

A method for predicting, diagnosing and prognosticating a neurodegenerative disease, such as Alzheimer's disease (AD), Mild Cognitive Impairment (MCI) and neurodegeneration in Down's syndrome (NDS) using glutaminyl cyclase (QC) as a diagnostic/prognostic indicator. The use of antibodies binding to QC and kits for performing said diagnostic method are also provided.

### CROSS-REFERENCE TO RELATED APPLICATIONS

This application claims priority to U.S. Provisional Application Ser. No. 61/085,154 filed on Jul. 31, 2008, which is incorporated herein by reference in its entirety.

### INCORPORATION-BY-REFERENCE OF MATERIAL SUBMITTED IN COMPUTER READABLE FORMAT (CRF)

The Sequence Listing, which is a part of the present disclosure, includes a computer readable form comprising nucleotide and/or amino acid sequences of the present invention. The subject matter of the Sequence Listing is incorporated herein by reference in its entirety.

### FIELD OF THE INVENTION

The present invention relates to a method for predicting, diagnosing and prognosticating a neurodegenerative disease, such as Alzheimer's disease (AD), Mild Cognitive Impairment (MCI) and neurodegeneration in Down's syndrome (NDS) using glutaminyl cyclase (QC) as a diagnostic/prognostic indicator.

### BACKGROUND OF THE INVENTION

Alzheimer Disease (AD) is a neurodegenerative disease that causes dementia. The terms “Alzheimer Disease” and “Alzheimer's Disease” are both utilized in the art, these terms being equivalent and are used interchangeably here and elsewhere. The period from first detection of AD to termination can range from a few years to 15 years, during which time the patient progressively suffers loss of both mental function and control of bodily functions. There is significant variability in the progress of the disease. While the majority of patients have a gradual, inexorable progression (losing on average 3 to 4 points on the 30 point Folstein mini-mental state score annually), approximately 30% of AD cases have a prolonged stable initial plateau phase lasting several years (Haxby J. V., et al., Individual trajectories of cognitive decline in patients with dementia of the Alzheimer type, J. Clin. Exp. Neuropsychol 14:575-592, 1992.). A subgroup of patients has a fulminant, rapidly progressive downhill course over several years (Mann, U., et al., Heterogeneity in Alzheimer's disease: Progression rate segregated by distinct neuropsychological and cerebral metabolic profiles, J. Neurol. Neurosurg. Psychiatry 55:956-959, 1992). Other patients (about 10% of cohorts) remain slowly progressive, showing only gradual decline from year to year (Grossi, D., et al., Senile dementias, II International Symposium (pp. 97-99), Paris: John Libbey Eurotext, 1988.). The pathological, chemical and molecular bases of this heterogeneity remain undetermined. Recognition of the variability of AD progression represents an important clinical insight, and may explain the diagnostic difficulties presented by “atypical” cases. While in certain cases, there is a familial manifestation of the AD disease, it appears that the majority of AD cases are non-familial, and until recently (see below), no simple biological marker for the disease had been determined.

Current methods used to diagnose AD involve analysis of cerebrospinal fluid (CSF) or brain tissue obtained from postmortem patients. Thus, among the markers currently under consideration are those related to the proteins, which account for the features found in Alzheimer brains postmortem. The neurofibrillary tangle is composed primarily of a hyperphosphorylated tau protein, a cytoskeletal protein. The neuritic plaque contains a core of amyloid protein, much of which is a 42-amino acid peptide (Aβ₄₂) derived from proteolytic cleavage of a larger precursor protein. Another form of this protein derived from the same precursor contains only 40 amino acids (Aβ₄₀). Deposits of this protein are found in the brains of Aβ victims. However, alterations in tau and the aforementioned beta amyloid peptides do not occur with sufficient frequency and magnitude so as to afford diagnostic value and therefore, blood tests based on these proteins do not seem to correlate well with AD. In addition to C-terminal variability, N-terminally modified Aβ peptides are abundant (Saido, T. C. et al. Dominant and differential deposition of distinct beta-amyloid peptide species, Aβ N3(pE), in senile plaques. Neuron 14, 457-466 (1995); Russo, C. et al. Presenilin-1 mutations in Alzheimer's disease. Nature 405, 531-532 (2000); Saido, T. C., Yamao, H., Iwatsubo, T. & Kawashima, S. Amino- and carboxyl-terminal heterogeneity of beta-amyloid peptides deposited in human brain. Neurosci. Lett. 215, 173-176 (1996)). It appears that a major proportion of the Aβ peptides undergoes N-terminal truncation by two amino acids, exposing a glutamate residue, which is subsequently cyclized into pyroglutamate (pE), resulting in Aβ3(pE)-42 peptides (Saido, T. C. et al. Dominant and differential deposition of distinct beta-amyloid peptide species, Aβ N3(pE), in senile plaques. Neuron 14, 457-466 (1995); Saido, T. C., Yamao, H., Iwatsubo, T. & Kawashima, S. Amino- and carboxyl-terminal heterogeneity of beta-amyloid peptides deposited in human brain. Neurosci. Lett. 215, 173-176 (1996)). Alternatively, pE may be formed following β′-cleavage by BACE1, resulting in Aβ N11(pE)-42 (Naslund, J. et al. Relative abundance of Alzheimer Aβ amyloid peptide variants in Alzheimer disease and normal aging. Proc. Natl. Acad. Sci. U.S.A. 91, 8378-8382 (1994); Liu, K. et al. Characterization of Aβ11-40/42 peptide deposition in Alzheimer's disease and young Down's syndrome brains: implication of N-terminally truncated Abeta species in the pathogenesis of Alzheimer's disease. Acta Neuropathol. 112, 163-174 (2006)). In particular Aβ N3(pE)-42 has been shown to be a major constituent of Aβ deposits in sporadic and familial AD (Saido, T. C. et al. Dominant and differential deposition of distinct beta-amyloid peptide species, Aβ N3(pE), in senile plaques. Neuron 14, 457-466 (1995); Miravalle, L. et al. Amino-terminally truncated Aβ peptide species are the main component of cotton wool plaques. Biochemistry 44, 10810-10821 (2005)).

The Aβ N3pE-42 peptides coexist with Aβ 1-40/1-42 peptides (Saido, T. C. et al. Dominant and differential deposition of distinct beta-amyloid peptide species, Abeta N3pE, in senile plaques. Neuron 14, 457-466 (1995); Saido, T. C., Yamao, H., Iwatsubo, T. & Kawashima, S. Amino- and carboxyl-terminal heterogeneity of beta-amyloid peptides deposited in human brain. Neurosci. Lett. 215, 173-176 (1996)), and, based on a number of observations, could play a prominent role in the pathogenesis of AD. For example, a particular neurotoxicity of Aβ N3pE-42 peptides has been outlined (Russo, C. et al. Pyroglutamate-modified amyloid beta-peptides—AbetaN3(pE)—strongly affect cultured neuron and astrocyte survival. J. Neurochem. 82, 1480-1489 (2002) and the pE-modification of N-truncated Aβ peptides confers resistance to degradation by most aminopeptidases as well as Aβ-degrading endopeptidases (Russo, C. et al. Pyroglutamate-modified amyloid beta-peptides—AbetaN3(pE)—strongly affect cultured neuron and astrocyte survival. J. Neurochem. 82, 1480-1489 (2002); Saido, T. C. Alzheimer's disease as proteolytic disorders: anabolism and catabolism of beta-amyloid. Neurobiol. Aging 19, S69-S75 (1998)). The cyclization of glutamic acid into pE leads to a loss of N-terminal charge resulting in accelerated aggregation of Aβ N3pE compared to the unmodified Aβ peptides (He, W. & Barrow, C. J. The Aβ 3-pyroglutamyl and 11-pyroglutamyl peptides found in senile plaque have greater beta-sheet forming and aggregation propensities in vitro than full-length Aβ. Biochemistry 38, 10871-10877 (1999); Schilling, S. et al. On the seeding and oligomerization of pGlu-amyloid peptides (in vitro). Biochemistry 45, 12393-12399 (2006)). Thus, reduction of Aβ N3pE-42 formation should destabilize the peptides by making them more accessible to degradation and would, in turn, prevent the formation of higher molecular weight Aβ aggregates and enhance neuronal survival.

However, for a long time it was not known how the pE-modification of Aβ peptides occurs. The present Applicant discovered that glutaminyl cyclase (QC) is capable to catalyze Aβ N3pE-42 formation under mildly acidic conditions, that specific QC inhibitors prevent Aβ N3pE-42 generation in vitro and that, therefore, inhibition of glutaminyl cyclase is a novel therapeutic concept for the causative treatment of Alzheimer's disease (Schilling, S., Hoffmann, T., Manhart, S., Hoffmann, M. & Demuth, H.-U. Glutaminyl cyclases unfold glutamyl cyclase activity under mild acid conditions. FEBS Lett. 563, 191-196 (2004); Cynis, H. et al. Inhibition of glutaminyl cyclase alters pyroglutamate formation in mammalian cells. Biochim. Biophys. Acta 1764, 1618-1625 (2006); Schilling et al. Inhibition of glutaminyl cyclase—a novel therapeutic concept for the causative treatment of Alzheimer's disease. Nature Medicine 14, 1106-1111 (2008)).

At present, there appears to be no satisfactory-diagnostic marker for existing AD, or for a subject, who although exhibiting normal cognitive responses, will inevitably, or most likely, develop AD.

Age-Associated Cognitive Decline (AACD) and Mild Cognitive Impairment (MCI) are terms used to identify individuals who experience a cognitive decline that falls short of dementia. These terms are equivalent, MCI being a more recently adopted term, and are used interchangeably throughout this application. Satisfaction of criteria (World Health Organization) for this diagnosis requires a report by the individual or family of a decline in cognitive function, which is gradual, and present at least 6 months. There may be difficulties across any cognitive domains (although memory is impaired in the vast majority of cases), and these must be supported by abnormal performance on quantitative cognitive assessments for which age and education norms are available for relatively healthy individuals (i.e., the patient is compared to normal subjects his/her own age). Performance must be at least 1 SD below the mean value for the appropriate population on such tests. Neither dementia, nor significant depression or drug effects may be present. No cerebral or systemic disease or condition known to cause cerebral cognitive dysfunction may be present. In Applicant's experience, all patients who were classified as CDR.5 (“questionable dementia”) on the Clinical Dementia rating scale and who met these exclusions, also met the criteria for AACD/MCI. About ⅓ of Alzheimer's patients have had a clearly definable period of isolated memory deficit which preceded their more global cognitive decline. (Haxby J. V., et al., Individual trajectories of cognitive decline in patients with dementia of the Alzheimer type, J. Clin. Exp. Neuropsychology 14:575-592, 1992.) Using AACD/MCI criteria, which look at other domains in addition to memory, the percentage with an identifiable prodrome is likely higher. Fortunately, not all AACD/MCI individuals seem to decline. It appears that a significant number of these subjects show a stable, non-progressive memory deficit on testing.

Attempts at predicting the onset of AD, MCI or NDS, or monitoring their progression have met with limited success.

### SUMMARY OF THE INVENTION

It has been discovered by the inventors of this application that an amount of QC in a biological sample obtained from a subject that deviates from a reference amount in a control person can be positively correlated to a neurological disease state. Thus, the correlation of the presence of QC with the disease state represents a positive and more direct test for diagnosis in a patient suffering from one of the neurodegenerative diseases described above.

Accordingly, the invention provides an easily administered biological sample test for predicting, diagnosing, or prognosticating AD, MCI and NDS using QC as a diagnostic marker.

The present invention is based at least in part on the discovery that an amount of glutaminyl cyclase (QC) in a biological sample obtained from a subject suffering from AD or MCI is elevated compared to an amount of QC in the biological sample obtained from a normal (i.e. healthy) control subject.

The indication that the amount of QC differs between these neurological diseases and normal controls, forms the basis for the development of a test for diagnosing AD, MCI or NDS in a subject. As such, the methods for diagnosing AD, MCI or NDS of the present invention by measuring the amount of QC in patient sample will greatly improve current clinical diagnostic assessment for patients suffering from these neurodegenerative diseases.

Based on the newly discovered differences in the amount of QC present in a biological sample obtained from a patient compared to that of a normal control, a strong correlation of the amount of QC can be made to a probable diagnosis of a neurodegenerative disease. A statistically significant elevation in the amount of QC relative to control samples is reasonably predictive that the patient has AD, NDS or MCI. A normal amount of QC as determined by an amount of QC characteristic of a control QC sample isolated from a normal age-matched population indicates that the patient does not have a neurodegenerative disease, such as AD, MCI or NDS. A positive indication of a neurodegenerative disease based on an elevated or reduced amount of QC in a biological sample relative to a normal control is generally considered together with other factors in making a definitive determination of a particular disease. Therefore, the elevated or reduced QC levels of the subject being tested will usually be considered together with other accepted clinical symptoms of AD, MCI or NDS-related conditions in making a determinative diagnosis of a neurodegenerative disease.

Thus, according to a first aspect of the invention, there is provided a method for diagnosing probable Alzheimer's Disease (AD), Neurodegeneration in Down's syndrome (NDS) or Mild Cognitive Impairment (MCI) in a subject, the method comprising: (a) detecting the amount of glutaminyl cyclase (QC), or its isoforms, in a biological sample obtained from said subject; and (b) comparing the detected amount of QC in the biological sample with an amount of QC characteristic of a normal control; whereby an elevated amount of QC in said biological sample relative to the normal control is a positive indicator of AD or MCI.

According to a preferred embodiment of the invention, the biological sample is a fluid body sample such as serum, plasma, urine or cerebrospinal fluid. More preferably, the fluid body sample is plasma.

According to a further embodiment of the present invention, the amount of QC is detected either on the basis of the QC protein level or the QC mRNA level.

The amount of QC detected or quantified in a biological sample from a subject can be accomplished by any means known in the art. Such means may include, but are not limited to, for example by immunoturbidimetric assay, immunofluorescence, immunodiffusion, enzyme-linked immunosorbent assay (ELISA), radioimmunoassay (RIA), Western Blot, protein activity assay or, for the determination of the QC mRNA level, Northern Blot or polymerase chain reaction (PCR) analysis, for example real-time PCR. Also useful are high performance liquid chromatography (HPLC), mass spectrometry (MS) and gas chromatography (GC), as well as their various configurations, including gas chromatograph-mass spectrometry (GC-MS), liquid chromatography-mass spectrometry (LC-MS) and liquid-chromatography-tandem mass spectrometry (LC-MS/MS) systems.

Preferably, the amount of QC in the biological sample is detected using an antibody that binds to QC in an immunoassay format. Thus, according to a preferred embodiment of the invention, there is provided a method of diagnosing a neurodegenerative disease in a subject, the method comprising: (a) obtaining a biological sample from said subject; (b) contacting said biological sample with an antibody that binds to glutaminyl cyclase (QC), or its isoforms; (c) allowing the antibody and QC to form an immune complex; and (d) detecting the amount of immune complex formed as an indication of the amount of QC in said biological sample; and (e) comparing the detected amount to a normal control; whereby a detected amount that is elevated or reduced relative to the normal control is a positive indicator of a neurodegenerative disease.

According to yet a further aspect of the invention, there is provided a diagnostic kit for determining whether a subject is suffering from a neurodegenerative disease comprising an antibody that binds to QC and an established standard of an amount of QC characteristic of a normal control. Reagents and instructions for carrying out the assays may also be included.

Other objects and features will be in part apparent and in part pointed out hereinafter.

### DESCRIPTION OF THE DRAWINGS

Those of skill in the art will understand that the drawings, described below, are for illustrative purposes only. The drawings are not intended to limit the scope of the present teachings in any way.

FIG. 1: FIG. 1(a) shows the analysis of QC transcript levels applying quantitative RT-PCR. Total RNA from human neocortical brain samples (Brodmann area 22) was isolated from normally aged and AD brains of different Braak stages as indicated. The QC transcript level was normalized to house-keeping transcript concentration. FIG. 1(b) shows the Western-Blot analysis for QC from the same cases and brain region as used for QC mRNA analysis. The extraction of soluble protein was normalized to the tissue weight. FIG. 1(c) shows the quantification of Aβ N3(pE)-42 (indicated as Aβ_{3(PE)-42}) and of Aβ 1-42 (Aβ₁₋₄₂) concentrations from the same cases and brain region applying ELISA analysis of SDS- and formic acid extracts of human neocortical brain samples. Note the robust increase in Aβ N3(pE)-42 peptide concentrations at early AD stages compared to the much more moderate increase in Aβ 1-42 peptides. FIG. 1(d) shows the immunohistochemical detection of total Aβ peptides by the antibody 4G8 and of Aβ N3(pE)-42 peptides in Brodmann area 22 from normally aged subjects and different AD stages. Sparse Aβ plaques were detected in normal aging but these deposits lacked Aβ N3(pE)-42 immunoreactivity. At all AD stages, however, the majority of Aβ plaques contains Aβ N3(pE)-42 peptides.

FIG. 2 shows the results of the determination of the gene expression rate of QC and CCL2 in stimulated THP-1 cells.

FIG. 3 shows the results of the determination of the specific QC activity in conditioned medium of THP-1 cells.

SEQUENCES OF AMYLOID PEPTIDES AND CHEMOKINES

| | |
|---|---|
| Aβ(1-42) | Asp-Ala-Glu-Phe-Arg-His-Asp-Ser- |
| (SEQ ID NO: 6) | Gly-Tyr-Glu-Val-His-His-Gln-Lys- |
| | Leu-Val-Phe-Phe-Ala-Glu-Asp-Val- |
| | Gly-Ser-Asn-Lys-Gly-Ala-Ile-Ile- |
| | Gly-Leu-Met-Val-Gly-Gly-Val-Val- |
| | Ile-Ala |
| Aβ(1-40) | Asp-Ala-Glu-Phe-Arg-His-Asp-Ser- |
| (SEQ ID NO: 7) | Gly-Tyr-Glu-Val-His-His-Gln-Lys- |
| | Leu-Val-Phe-Phe-Ala-Glu-Asp-Val- |
| | Gly-Ser-Asn-Lys-Gly-Ala-Ile-Ile- |
| | Gly-Leu-Met-Val-Gly-Gly-Val-Val |
| Aβ(3-42) | Glu-Phe-Arg-His-Asp-Ser-Gly-Tyr- |
| (SEQ ID NO: 8) | Glu-Val-His-His-Gln-Lys-Leu-Val- |
| | Phe-Phe-Ala-Glu-Asp-Val-Gly-Ser- |
| | Asn-Lys-Gly-Ala-Ile-Ile-Gly-Leu- |
| | Met-Val-Gly-Gly-Val-Val-Ile-Ala |
| Aβ(3-40) | Glu-Phe-Arg-His-Asp-Ser-Gly-Tyr- |
| (SEQ ID NO: 9) | Glu-Val-His-His-Gln-Lys-Leu-Val- |
| | Phe-Phe-Ala-Glu-Asp-Val-Gly-Ser- |
| | Asn-Lys-Gly-Ala-Ile-Ile-Gly-Leu- |
| | Met-Val-Gly-Gly-Val-Val |
| Aβ(1-38) | Asp-Ala-Glu-Phe-Arg-His-Asp-Ser- |
| (SEQ ID NO: 10) | Gly-Tyr-Glu-Val-His-His-Gln-Lys- |
| | Leu-Val-Phe-Phe-Ala-Glu-Asp-Val- |
| | Gly-Ser-Asn-Lys-Gly-Ala-Ile-Ile- |
| | Gly-Leu-Met-Val-Gly-Gly |
| Aβ(3-38) | Glu-Phe-Arg-His-Asp-Ser-Gly-Tyr- |
| (SEQ ID NO: 11) | Glu-Val-His-His-Gln-Lys-Leu-Val- |
| | Phe-Phe-Ala-Glu-Asp-Val-Gly-Ser- |
| | Asn-Lys-Gly-Ala-Ile-Ile-Gly-Leu- |
| | Met-Val-Gly-Gly |
| ABri | EASNCFAIRHFENKFAVETLICSRTVKKNIIE |
| (SEQ ID NO: 12) | EN |
| ADan | EASNCFAIRHFENKFAVETLICFNLFLNSQEK |
| (SEQ ID NO: 13) | HY |
| CCL2 | QPDAINAPVTCCYNFTNRKISVQRLASYRRIT |
| (small inducible | SSKCPKEAVIFKTIVAKEICADPKQKWVQDSM |
| cytokine A2) | DHLDKQTQTPKT |
| (SEQ ID NO: 14) | |
| Swiss-Prot: P13500 | |
| CCL7 | QPVGINTSTTCCYRFINKKIPKQRLESYRRTT |
| (Small-inducible | SSHCPREAVIFKTKLDKEICADPTQKWVQDFM |
| cytokine A7) | KHLDKKTQTPKL |
| (SEQ ID NO: 15) | |
| Swiss-Prot: P80098 | |
| CCL8 | QPDSVSIPITCCFNVINRKIPIQRLESYTRIT |
| small inducible | NIQCPKEAVIFKTKRGKEVCADPKERWVRDSM |
| cytokine A8) | KHLDQIFQNLKP |
| (SEQ ID NO: 16) | |
| Swiss-Prot: P80075 | |
| CCL9/10 | QITHATETKEVQSSLKAQQGLEIEMFHMGFQD |
| (Small-inducible | SSDCCLSYNSRIQCSRFIGYFPTSGGCTRPGI |
| cytokine A9) | IFISKRGFQVCANPSDRRVQRCIERLEQNSQP |
| (SEQ ID NO: 17) | RTYKQ |
| Swiss-Prot: P51670 | |
| CCL13 | QPDALNVPSTCCFTFSSKKISLQRLKSYVITT |
| (Small-inducible | SRCPQKAVIFRTKLGKEICADPKEKWVQNYMK |
| cytokine A13) | HLGRKAHTLKT |
| (SEQ ID NO: 18) | |
| Swiss-Prot: Q99616 | |
| CCL15 | QFINDAETELMMSKLPLENPVVLNSFHFAADC |
| (Small-inducible | CTSYISQSIPCSLMKSYFETSSECSKPGVIFL |
| cytokine A15) | TKKGRQVCAKPSGPGVQDCMKKLKPYSI |
| (SEQ ID NO: 19) | |
| Swiss-Prot: Q16663 | |
| CCL16 | QPKVPEWVNTPSTCCLKYYEKVLPRRLWGYRK |
| (Small-inducible | ALNCHLPAIIFVTKRNREVCTNPNDDWVQEYI |
| cytokine A16) | KDPNLPLLPTRNLSTVKIITAKNGQPQLLNSQ |
| (SEQ ID NO: 20) | |
| Swiss-Prot: O15467 | |
| Fractalkine | QHHGVTKCNITCSKMTSKIPVALLIHYQQNQA |
| (neurotactin) | SCGKRAIILETRQHRLFCADPKEQWVKDAMQH |
| (SEQ ID NO: 21) | LDRQAAALTRNGGTFEKQIGEVKPRTTPAAGG |
| Swiss-Prot: P78423 | MDESVVLEPEATGESSSLEPTPSSQEAQRALG |
| | TSPELPTGVTGSSGTRLPPTPKAQDGGPVGTE |
| | LFRVPPVSTAATWQSSAPHQPGPSLWAEAKTS |
| | EAPSTQDPSTQASTASSPAPEENAPSEGQRVW |
| | GQGQSPRPENSLEREEMGPVPAHTDAFQDWGP |
| | GSMAHVSVVPVSSEGTPSREPVASGSWTPKAE |
| | EPIHATMDPQRLGVLITPVPDAQAATRRQAVG |
| | LLAFLGLLFCLGVAMFTYQSLQGCPRKMAGEM |
| | AEGLRYIPRSCGSNSYVLVPV |
| CCL25 | QGVFEDCCLAYHYPIGWAVLRRAWTYRIQEVS |
| (Small-inducible | GSCNLPAAIFYLPKRHRKVCGNPKSREVQRAM |
| cytokine A25) | KLLDARNKVFAKLHHNTQTFQAGPHAVKKLSS |
| (SEQ ID NO: 22) | GNSKLSSSKFSNPISSSKRNVSLLISANSGL |
| Swiss-Prot: O15444 | |

### DETAILED DESCRIPTION OF THE PRESENTLY PREFERRED EMBODIMENTS

The present invention provides an efficient and rapid in vitro method for diagnosing a neurodegenerative disease by directly detecting an amount of QC in a biological sample obtained from a subject and comparing the detected amount of QC with an amount of QC characteristic of a normal control. An elevated amount of QC in the biological sample of the subject is a positive indication of AD or MCI or NDS. Thus, as described herein, it is demonstrated that QC is consistently and significantly elevated in a biological sample of AD, NDS or MCI patients compared to normal controls. As such, the methods for diagnosing AD, MCI or NDS of the present invention by detecting or quantifying the amount of QC in a patient sample will greatly improve current clinical diagnostic assessment for patients suffering from these neurodegenerative diseases.

Accordingly, there is provided a method for assessing whether a subject may be suffering from AD, MCI or NDS using QC as a biological marker.

Glutaminyl cyclase or glutaminyl-peptide cyclotransferase (QC, EC 2.3.2.5) catalyzes the intramolecular cyclization of N-terminal glutaminyl residues into pyroglutamic acid (5-oxo-proline, pGlu*) under liberation of ammonia and the intramolecular cyclization of N-terminal glutamyl residues into pyroglutamic acid under liberation of water.

A QC was first isolated by Messer from the Latex of the tropical plant Carica papaya in 1963 (Messer, M. 1963 Nature 4874, 1299). 24 years later, a corresponding enzymatic activity was discovered in animal pituitary (Busby, W. H. J. et al. 1987 J Biol Chem 262, 8532-8536; Fischer, W. H. and Spiess, J. 1987 Proc Natl Acad Sci USA 84, 3628-3632). For the mammalian QCs, the conversion of Gln into pGlu by QC could be shown for the precursors of TRH and GnRH (Busby, W. H. J. et al. 1987 J Biol Chem 262, 8532-8536; Fischer, W. H. and Spiess, J. 1987 Proc Natl Acad Sci USA 84, 3628-3632). In addition, initial localization experiments of QC revealed a co-localization with its putative products of catalysis in the bovine tractus hypothalamo-hypophysalisfurther improving the suggested function in peptide hormone maturation (Bockers, T. M. et al. 1995 J Neuroendocrinol 7, 445-453). In contrast, the physiological function of the plant QC is less clear. In case of the enzyme from C. papaya, a role in the plant defence against pathogenic microorganisms was suggested (El Moussaoui, A. et al. 2001 Cell Mol Life Sci 58, 556-570). Putative QCs from other plants were identified by sequence comparisons recently (Dahl, S. W. et al. 2000 Protein Expr Purif 20, 27-36). The physiological function of these enzymes, however, is still ambiguous.

The QCs known from plants and animals show a strict specificity for L-Glutamine in the N-terminal position of the substrates and their kinetic behaviour was found to obey the Michaelis-Menten equation (Pohl, T. et al. 1991 Proc Natl Acad Sci USA 88, 10059-10063; Consalvo, A. P. et al. 1988 Anal Biochem 175, 131-138; Gololobov, M. Y. et al. 1996 Biol Chem Hoppe Seyler 377, 395-398). A comparison of the primary structures of the QCs from C. papaya and that of the highly conserved QC from mammals, however, did not reveal any sequence homology (Dahl, S. W. et al. (2000) Protein Expr Purif 20, 27-36). Whereas the plant QCs appear to belong to a new enzyme family (Dahl, S. W. et al. (2000) Protein Expr Purif 20, 27-36), the mammalian QCs were found to have a pronounced sequence homology to bacterial aminopeptidases (Bateman, R. C. et al. 2001 Biochemistry 40, 11246-11250), leading to the conclusion that the QCs from plants and animals have different evolutionary origins.

Gostranova et al. have found that glutaminyl cyclase activity is a characteristic feature of cerebrospinal fluid in multiple sclerosis patients and controls (Gostranova et al., Clin Chim Acta. 2008 389 (1-2), pp. 152-159).

Different isoforms of QC, the glutaminyl-peptide cyclotransferase-like proteins (QPCTLs) have been observed (WO 2008/034891). These novel proteins have significant sequence similarity to glutaminyl cyclase, e.g. the QPCTL from human (further named as isoQC) (GenBank accession no. NM_{—}017659).

Multiple isoforms of a protein, such as QC or human isoQC, can also be produced from a single gene by a variety of mechanisms, including alternative RNA splicing, post-translational proteolytic processing and cell type-specific glycosylation. Thus, the terms “glutaminyl cyclase”, “QC” and “isoQC” as used herein refer to QC in its native form, as well as any of its isoforms.

Preferred for the use of the present invention are human QC or its isoforms, having an amino acid sequence selected from the group of SEQ ID NO's: 1, 2, 3, 4 and 5.

More preferred for use in the methods of the present invention is the human QPCTL having an amino acid sequence of SEQ ID NO. 2, or even preferred of SEQ ID NO: 3.

Even preferred for use in the methods of the present invention are spliceforms of human QPCTL having an amino acid sequence of SEQ ID NO. 4 or of SEQ ID NO: 5.

Most preferred for use in the methods of the present invention is human QC having the amino acid sequence of SEQ ID NO: 1.

Thus, according to a first aspect of the present invention, there is provided a method for diagnosing probable Alzheimer's Disease (AD), Neurodegeneration in Down's Syndrome (NDS) or Mild Cognitive Impairment (MCI) in a subject, the method comprising:

(a) detecting the amount of glutaminyl cyclase (QC), or an isoform thereof, in a biological sample obtained from said subject; and

(b) comparing the detected amount of QC in the biological sample with an amount of QC characteristic of a normal control;

whereby an elevated amount of QC in said biological sample relative to the normal control is a positive indicator of AD, NDS or MCI.

It has been demonstrated by inventors of the present invention that an elevated amount of QC in a biological sample may correlate with an elevated amount of N-terminally truncated and pyroglutamated amyloid beta peptides, such as for example Aβ N3pE-42 and/or Aβ N3pE-40 and/or Aβ N3pE-38.

Thus, according to a further aspect of the present invention, there is provided a method for diagnosing probable Alzheimer's Disease (AD), Neurodegeneration in Down's Syndrome (NDS) or Mild Cognitive Impairment (MCI) in a subject, the method comprising:

(a) detecting the amount of glutaminyl cyclase (QC), or an isoform thereof, in a biological sample obtained from said subject; and

(b) further detecting the amount of Aβ N3pE-X,

(c) comparing the detected amount of QC and Aβ N3pE-X in the biological sample with an amount of QC and Aβ N3pE-X characteristic of a normal control;

whereby an elevated amount of QC and Aβ N3pE-X in said biological sample relative to the normal control is a positive indicator of AD, NDS or MCI, and

wherein X is an integer selected from 38, 40 and 42.

In a preferred embodiment, X is 42.

In a further preferred embodiment, X is 40.

In a yet preferred embodiment, X is 38.

Further preferred are methods, wherein not only a single form of the N-terminally truncated and pyroglutamated amyloid beta peptides but a combination of Aβ N3pE-42 and/or Aβ N3pE-40 and/or Aβ N3pE-38 is detected together with QC.

Further preferred are methods, wherein not only a single form of the N-terminally truncated and pyroglutamated amyloid beta peptides but a combination of Aβ N3pE-42 and/or Aβ N3pE-40 and/or Aβ N3pE-38 and/or peptides occurring in familial Alzheimer's dementias, such as pGluABri or pGluADan, is detected together with QC.

“pGlu-A” or “Aβ N3pE” refers to N-terminally truncated forms of Aβ, that start at the glutamic acid residue at position 3 in the amino acid sequence of Aβ, and wherein said glutamic acid residue is cyclized to form a pyroglutamic acid residue. In particular, by pGlu-Aβ as used herein are meant those fragments which are involved in or associated with the amyloid pathologies including, but not limited to, pGlu-Aβ 3-38, pGlu-Aβ 3-40, p-Glu-Aβ 3-42.

It has further been demonstrated by the inventors of the present invention that an elevated amount of QC in a biological sample may correlate with an elevated amount of a chemokine, such as for example CCL2, CCL7, CCL8, CCL9/10, CCL13, CCL15, CCL16, CCL25 and Fractalkine.

Thus, according to a further aspect of the present invention, there is provided a method for diagnosing Alzheimer's Disease (AD), Neurodegeneration in Down's Syndrome (NDS) or Mild Cognitive Impairment (MCI) in a subject, the method comprising:

(a) detecting the amount of glutaminyl cyclase (QC), or an isoform thereof, in a biological sample obtained from said subject; and

(b) further detecting the amount of a chemokine,

(c) comparing the detected amount of QC and the chemokine in the biological sample with an amount of QC and the chemokine characteristic of a normal control;

whereby an elevated amount of QC and chemokine in said biological sample relative to the normal control is a positive indicator of AD, NDS or MCI.

In a preferred embodiment, said chemokine is of mammalian origin. More preferably, said chemokine is a human chemokine. Most preferably, said chemokine is human CCL2.

In a further preferred embodiment, any of the aforementioned methods for diagnosing Alzheimer's Disease (AD), Neurodegeneration in Down's Syndrome (NDS) or Mild Cognitive Impairment (MCI) may also be performed in vitro in a biological sample of a subject.

The term “subject” refers to a mammal which is afflicted with, or suspected to be afflicted with a neurogenerative disease such as AD, MCI or NDS. Preferably, “subject” refers to a human.

The term “biological sample” refers to any source of biological material, including, but are not limited to, peripheral blood, plasma, lymphocytes, cerebrospinal fluid, urine, saliva, epithelia, fibroblasts, or any other sample comprising QC protein.

In a preferred embodiment, the amount of QC is detected in a body fluid sample obtained from a mammal, most preferably a human. The term “body fluid” refers to all fluids that are present in the human body including but not limited to blood, lymph, urine and cerebrospinal fluid (CSF) comprising QC. The blood sample may include a plasma sample or a serum sample, or fractions derived from these samples. The sample can be treated prior to use, such as preparing plasma from blood, diluting viscous fluids, and the like. Preferably, the plasma sample is treated with an anti-coagulant, such as EDTA.

According to a preferred embodiment of the present invention, the amount of QC is detected in a blood sample taken from the subject, more preferably a plasma sample. Thus, the present invention preferably relates to a method as described above, comprising the steps of: obtaining a plasma sample from said subject; detecting the amount of QC in the plasma sample; comparing the detected amount of QC in the plasma sample with the amount of QC in a plasma sample from a normal control, whereby an elevated amount of QC relative to the normal control is a positive indication of AD, NDS or MCI. Elevated amounts of QC have been shown to correlate with and are useful in aiding the diagnosis of AD, NDS and MCI.

An “elevated amount” of QC (or an isoform thereof) means that the amount of QC detected in the samples of the subjects is greater than the mean amount of QC characteristic of a normal control person beyond the range of experimental error, as known in the art. Preferably, the amount of QC detected in the samples of the subjects is 10% greater than said mean amount of QC characteristic of a normal control person. More preferably, the amount of QC (or an isoform thereof) detected in the samples of the subjects is 25% greater, or, even more preferred 50% or 75% greater than said mean amount of QC characteristic of a normal control person. Most preferably, the amount of QC (or an isoform thereof) detected in the samples of the subjects is several times greater than said mean amount of QC characteristic of a normal control person, e.g. 2, 3, 4, 5, 6, 7, 8, 9, 10 or more times greater.

A “normal control” is a biological sample of the same type obtained from the subject, for example that is obtained from at least one normal age-matched control person or from the patient at another time. In an embodiment, the normal control is taken from the patient at an earlier time. A normal control sample from a normal age-matched population should be isolated from an adequate population sample of healthy age matched controls with no history of AD, MCI or NDS in their family. By way of example, a plasma QC level higher than the control levels of QC, as determined by an adequate control population sample size, is indicative of AD, NDS or MCI. One of skill in the art will appreciate that the sample from the subject to be diagnosed is assessed against a normal age-matched control and that a significant elevation or reduction in the amount of QC in the subject's protein sample is determined based on comparison to the controls used in the given assay.

According to a further embodiment of the present invention, the amount of QC, or an isoform thereof, is detected either on the basis of the protein level or the mRNA level of said QC or isoform thereof.

The amount of QC detected or quantified in a subject's biological sample can be accomplished by any means known in the art. Such means may include, but are not limited to, for example by immunoturbidimetric assay, immunofluorescence, immunodiffusion, enzyme-linked immunosorbent assay (ELISA), radioimmunoassay (RIA), Western Blot, protein activity assay, or, for the determination of the QC mRNA level, Northern Blot or polymerase chain reaction (PCR) analysis, for example real-time PCR. Also useful are high performance liquid chromatography (HPLC), mass spectrometry (MS) and gas chromatography (GC), as well as their various configurations, including gas chromatograph-mass spectrometry (GC-MS), liquid chromatography-mass spectrometry (LC-MS) and liquid-chromatography-tandem mass spectrometry (LC-MS/MS) systems, to name a few.

While detection of QC can be accomplished by methods known in the art for detecting peptides, the use of immmunological detection techniques using antibodies, antibody fragments, recombinant antibodies, and the like, is preferred. Therefore, such detection of QC includes, but is not limited to, the use of antibodies, which specifically bind to QC, or its isoforms, to form an immune complex, as well as reagents for detecting the formation of the immune complex. Particularly suitable detection techniques employing one or more antibodies include immunoturbidimetric assay, immunofluorescence, immunodiffusion, ELISA, RIA and the like.

Such antibodies may be polyclonal or monoclonal. Methods to produce polyclonal or monoclonal antibodies are well known in the art. For a review, see Harlow and Lane (Harlow, E. and Lane, D., Antibodies: A Laboratory Manual, Cold Spring Harbor Laboratory Press, Cold Spring Harbor, N.Y., 1988) and Yelton et al. (Yelton D. E. and Scharff M. D. Monoclonal Antibodies: a powerful new tool in biology and medicine. Ann. Rev. Biochem. 50:657-680, 1981), both of which are herein incorporated by reference. For monoclonal antibodies, see Kohler and Milstein (Kohler G. and Milstein C, Continuous cultures of fused cells secreting antibody of predefined specificity, Nature 256:495-497, 1975), herein incorporated by reference. The antibodies of the invention are of any isotype, e.g., IgG or IgA, and polyclonal antibodies are of a single isotype or a mixture of isotypes.

According to a preferred embodiment of the invention, the anti-QC antibody is a monoclonal antibody. Although anti-QC antibodies are widely commercially available, antibodies for use in the various immunoassays described herein, can be produced according to standard methods.

Further, the monoclonal anti-QC antibody is capable of recognizing QC in its native form, as well as any of its isoforms. Thus, any monoclonal antibody that specifically recognizes QC, including its isoforms, can be used in said method for the quantification of QC.

Preferred are monoclonal antibodies, that specifically recognize QC but show low, or more preferably, no crossreactivity with isoforms of QC. Alternatively preferred are monoclonal antibodies that specifically recognize a particular isoform of QC but show low, or more preferably, no crossreactivity with QC.

Suitable anti-QC antibodies are, for example, those which are commercially available from Abnova (Taipei City, Taiwan), e.g. a mouse polyclonal antibody (Cat. #H00025797-B01P) and a rabbit polyclonal antibody (Cat. #H00025797-D01P).

A suitable anti-QPCTL antibody is, for example, the commercially available mouse polyclonal antibody from Abnova (Taipei City, Taiwan, Cat. #H00054814-B01P).

Also fragments derived from these monoclonal antibodies such as Fab, F(ab)_{2/} ssFv (single chain variable fragment) and other antibody-like constructs that retain the variable region of the antibody, providing they have retained the original binding properties, can be used in a method of the present invention. Such fragments are commonly generated by, for instance, enzymatic digestion of the antibodies with papain, pepsin, or other proteases. It is well known to the person skilled in the art that monoclonal antibodies, or fragments thereof, can be modified for various uses. Thus, antibodies of the invention, may be recombinant, e.g., chimeric (e.g., constituted by a variable region of murine origin associated with a human constant region), humanized (a human immunoglobulin constant backbone together with hypervariable region of animal, e.g., murine, origin), and/or single chain.

An antibody specific for QC, or its isoforms, used in a method of the present invention may be labelled by an appropriate label and identified in the biological sample based upon the presence of the label. The label allows for the detection of the antibody when it is bound to QC. Examples of labels include, but are not limited to, the following: radioisotopes (e.g., ³H, ¹⁴C, ³⁵S, ¹²⁵I, ¹³¹I), fluorescent labels (e.g., FITC, rhodamine, lanthanide phosphors), luminescent labels, enzymatic labels (e.g., horseradish peroxidase, beta-galactosidase, luciferase, alkaline phosphatase), chemiluminescent, and biotinyl groups.

Methods for conjugating or labelling the antibodies discussed above may be readily accomplished by one of ordinary skill in the art (see for example Inman, “Methods In Enzymology”, Vol. 34, Affinity Techniques, Enzyme Purification: Part B, Jakoby and Wichek (eds.), Academic Press, New York, p. 30, 1974; and Wilchek and Bayer, “The Avidin-Biotin Complex in Bioanalytical Applications,” Anal. Biochem. 171:1-32, 1988).

For diagnostic applications, the anti-QC antibody is either in a free state or immobilized on a solid support, such as a tube, a bead, or any other conventional support used in the field. Immobilization is achieved using direct or indirect means. “Direct means” include passive adsorption (non-covalent binding) or covalent binding between the support and the reagent. By “indirect means” is meant that an anti-reagent compound that interacts with a reagent is first attached to the solid support. Indirect means may also employ a ligand-receptor system, for example, where a molecule such as a vitamin is grafted onto the reagent and the corresponding receptor immobilized on the solid phase. This is illustrated by the biotin-streptavidin system.

Those skilled in the art will readily understand that an immune complex is formed between QC in the biological sample and the antibody, and that any unbound material is removed prior to detecting the complex. It is understood that an antibody of the invention is used for quantifying an amount of QC in the biological sample, such as, for example, blood, plasma, lymphocytes, cerebrospinal fluid, urine, saliva, epithelia and fibroblasts.

As is known in the art, the determination of such antibody binding can be performed using a great variety of immunoassay formats including, but not limited to immunoturbidimetric assay (agglutination) , enzyme-linked immunosorbent assay (ELISA) and radioimmunoassay (RIA) (see, for example, “Principles and Practice of Immunoassay” (1991) Christopher P. Price and David J. Neoman (eds), Stockton Press, New York, N.Y. and Ausubel et al. (eds) (1987) in “Current Protocols in Molecular Biology” John Wiley and Sons, New York, N.Y., both of which are incorporated herein by reference). Detection may be by colormetic or radioactive methods or any other conventional methods known to one skill in the art. Other standard techniques known in the art are described in “Methods in Immunodiagnosis”, 2nd Edition, Rose and Bigazzi, eds., John Wiley and Sons, New York 1980 and Campbell et al.; “Methods of Immunology”, W. A. Benjamin, Inc., 1964; U.S. Pat. Nos. 4,366,241; 4,376,110; 4,517,288; and 4,837,168, the disclosures of which are incorporated herein by reference. For a review of the general immunoassays, see also “Methods In Cell Biology”, Vol. 37, Asai, ed. Academic Press, Inc. New York (1993); “Basic And Clinical Immunology” 7^{th} Edition, Stites & Terr, eds. (1991).

Such assays for detecting QC may be a direct, indirect, competitive, or noncompetitive immunoassay as described in the art (see, for example, “Principles and Practice of Immunoassay” (1991) Christopher P. Price and David J. Neoman (eds), Stockton Press, New York, N.Y.; Ausubel et al. (eds) (1987) in “Current Protocols in Molecular Biology” John Wiley and Sons, New York, N.Y.; and Oellirich, M. 1984. J. Clin. Chem. Clin. Biochem. 22: 895-904, incorporated herein by reference).

Noncompetitive immunoassays are assays in which the amount of QC is directly detected. In the “sandwich” assay, for example, the anti-QC antibodies can be bound directly to a solid substrate where they are immobilized. These immobilized antibodies then capture the QC present in the biological sample. The QC thus immobilized is then bound by a labeling agent, such as a second human QC antibody bearing a label.

In a competitive immunoassay, the amount of antigen present in the biological sample is determined indirectly following addition of a known amount of labeled antigen to the sample and detecting the amount of labeled antigen bound with antibodies. For example, a known amount of, in this case, labeled QC is added to the biological sample and the sample is then contacted with anti-QC antibodies. The amount of labeled QC bound to the anti-QC antibody is inversely proportional to the concentration of QC in the biological sample. This is because the greater the amount of labeled QC detected, the less the amount of QC was available in the biological sample to compete with the labeled QC.

Diagnostic kits for carrying out the assays for diagnosing AD, MCI or NDS in a subject are also provided. Thus, the present invention can be practiced using a diagnostic kit that includes at least one antibody specific for QC, and its isoforms, as described herein as well as any reagents necessary for the detection of antibody-QC binding immune complexes. Generally, the kit may include a single antibody that specifically recognizes QC, and its isoforms. On the other hand, the kit may include a primary antibody that specifically recognizes QC, and its isoforms, as well as a secondary antibody that is conjugated with a signal-producing label and is capable of binding to the primary antibody, or at a site different from the site where the primary antibody binds. The signal-producing label linked to the secondary antibody may be, but is not limited to, an enzyme, such as horseradish peroxidase or alkaline phosphatase. The kits may further comprise other reagents for carrying out the assay such as buffers, a solid support, solutions and the like. The kit may also contain instructions for carrying out the method of the invention using one or more antibodies in diagnostic assays.

In some embodiments, the numbers expressing quantities of ingredients, properties such as molecular weight, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

In some embodiments, the terms “a” and “an” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present invention.

Having described the invention in detail, it will be apparent that modifications, variations, and equivalent embodiments are possible without departing the scope of the invention defined in the appended claims. Furthermore, it should be appreciated that all examples in the present disclosure are provided as non-limiting examples.

### Examples

The following non-limiting examples are provided to further illustrate the present invention. It should be appreciated by those of skill in the art that the techniques disclosed in the examples that follow represent approaches the inventors have found function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

### Example 1

### Formation of Aβ N3PE-42 and QC Expression In Vivo

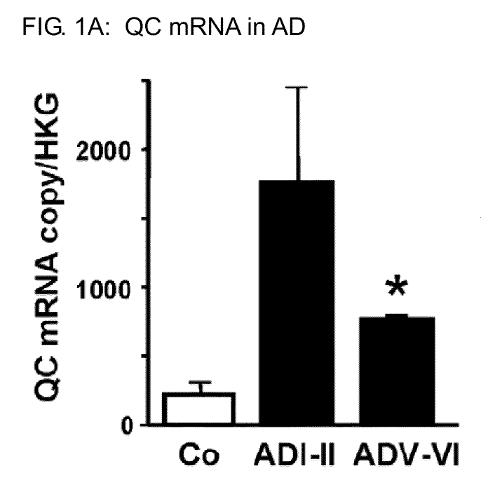

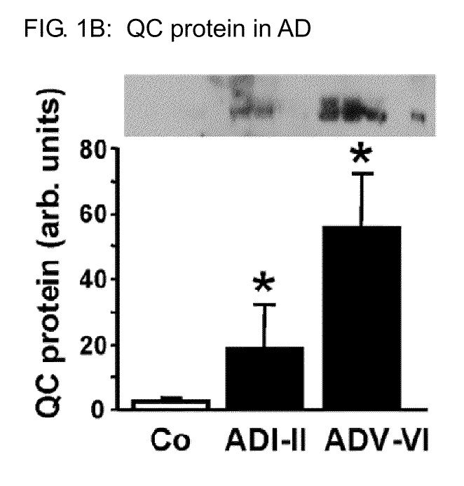

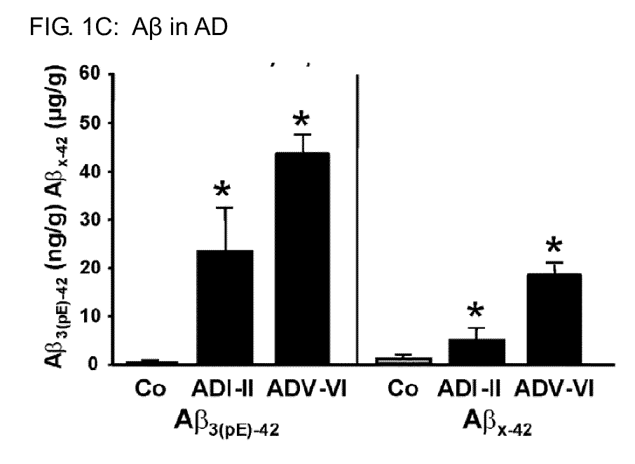

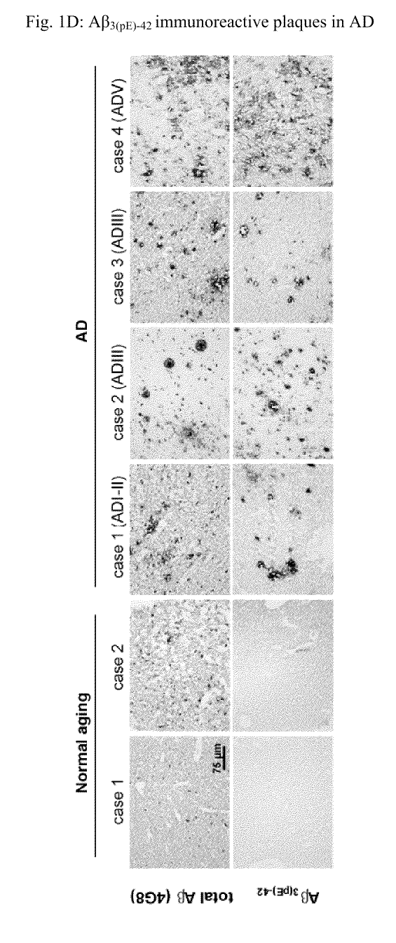

A widespread QC distribution has been detected in mammalian brain with considerable expression in hippocampus and cortex. In order to assess whether QC expression in AD can be correlated with generation of Aβ N3pE-42, QC mRNA and protein concentrations were analyzed in human neocortical brain samples post mortem (see e.g., FIG. 1a, b). Intriguingly, the inventors found an upregulation of QC mRNA and protein in AD brain samples, compared to normal aging. Moreover, significant concentrations of Aβ N3pE-42 were detected in samples from AD patients in contrast to non-demented individuals supporting a role of QC in generation of Aβ N3pE-42 (see e.g., FIG. 1c). On the other hand, ELISA analysis revealed high Aβ x-42 concentrations in normally aged control subjects and a much smaller increase at early AD stages (see e.g., FIG. 1c). This observation was corroborated by immunohistochemistry applying antibodies detecting total Aβ (4G8) or specifically Aβ N3pE-42 (see e.g., FIG. 1d). Conspicuous immunoreactivity for Aβ was detected in brain sections from all groups. In contrast, Aβ N3pE-42 staining was absent in normal aging but specific for AD brain tissue, where Aβ N3pE-42-immunoreactive plaque load was almost as high as the total of Aβ plaque density.

Human Brain Tissue

The definite diagnosis of AD for all cases used in this study was based on the presence of neurofibrillary tangles and neuritic plaques in the hippocampal formation and neocortical areas and met the criteria of the National Institute of Neurologic and Communicative Disorders and Stroke (NINDS) and the Alzheimer's Disease and Related Disorders Association (ADRDA). Cortical tissue (Brodmann area 22) from the same cases was used for the quantification of QC mRNA concentrations, QC protein and Aβ N3pE-42. In total, 10 control cases and 10 AD cases each of Braak staging I-II and V-VI were analyzed. The groups were matched for gender and age (control: mean 72 years±6.6 years; AD I-II: mean 73 years±3.1 years; AD V-VI: mean 77 years±6.6 years). The mean post morten interval (PMI) was similar among the groups and ranged from 26 to 96 hours. The duration of PMI was neither related to the detection of QC by Western blot analysis nor to quantification of Aβ by ELISA. For QC mRNA detection by qRT-PCR, only tissue samples with a PMI below 48 hours were included.

QC mRNA Quantification and QC Western Blot Analysis

Tissue samples were homogenized by means of the homogenizer Precellys with 1.4 mm ceramic beads (5000 rpm, 30 sec, peqlab). RNA was isolated using the NucleoSpin RNA II kit (Macherey Nagel) according to the manufacturer's instructions. Constant 100 ng of RNA were reverse transcribed to cDNA using random primers (Roche) and Superscript II (Invitrogen). Quantitative real-time PCR was performed in a Rotorgene3000 (Corbett Research) using the QuantiTect Primer Assay for QPCT (QT00013881, Qiagen) as well as the QuantiTect SYBR Green RT-PCR kit (Qiagen). Absolute amounts of QC were determined using six dilutions of the external QC standard DNA (full length QC cloned in the pcDNA3 vector) in duplicate. For verification of the PCR, product melting curves were generated and single amplicons were confirmed by agarose gel electrophoresis. Absolute amounts were determined with the Rotorgene software version 4.6 in quantitation mode. Normalization was done against the two most stably expressed housekeeping genes HPRT and GAPDH (geNorm). For Western-Blot analysis, the brain samples (50 mg) were homogenized in buffer (1 ml) containing 10 mM Tris pH 7.5, 100 mM NaCl, 5 mM EDTA and 0.5% Triton X-100 and 10% glycerol. The tissue was homogenized by several strokes in Downs-homogenizer and subjected to 3×10 s of ultrasonic shock. The resulting homogenate was cleared by centrifugation at 20000×g for 25 min. A total of 12 μg protein of each sample was separated in Tris-Glycine SDS-PAGE. QC was detected using purified rabbit polyclonal antibodies raised against recombinant human QC. For visualization, blot membranes were incubated with secondary antibody conjugated with horseradish peroxidase (Cell Signaling) in TBS-T containing 5% (w/v) dry milk and subsequently developed using the SuperSignal West Pico System (Pierce) according to the manufacturer's protocol.

### Example 2

### Determination of Gene Expression Rate of QC and CCL2 in Stimulated THP-1 Cells

Human monocytic leukaemia cell line THP-1 cells were cultivated in suspension (5×10⁵ cells per ml medium) in RPMI-1640 (Rosewell Park Memorial Institute Medium 1640 (Invitrogen)) containing 10% FCS (=FBS, Fetal Bovine Serum (Invitrogen)) and 60 μg/ml gentamycin (Invitrogen) at 37° C. in 5% CO₂ and 95% air humidified atmosphere.

To investigate stimulation effects of QC and CCL2 2×10⁶ cells were seeded in 24 well plates (Greiner) into 1 ml culture medium without FCS containing different concentrations of lipopolysaccharides (LPS; Sigma). After 24 h incubation the medium was removed from the cells by centrifugation (5 min 300×g).

RNA isolation was carried out with the Nucleo-Spin® RNA II Kit (Macherey & Nagel) followed by the determination of the RNA concentration. Using the SuperScript™ II Reverse Transcriptase Kit from Invitrogen 1 μg RNA was transcribed into cDNA.

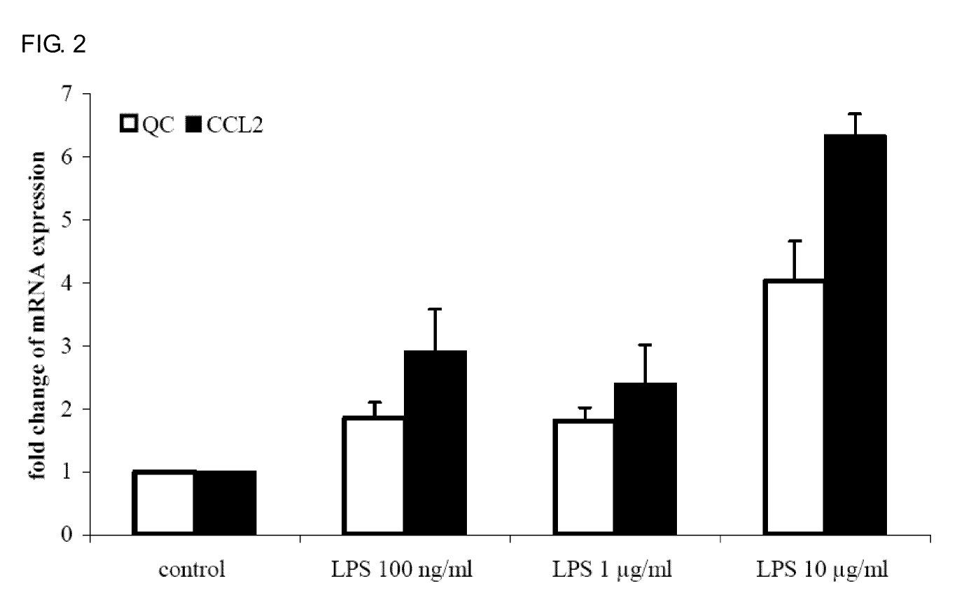

The gene expression rate of QC and CCL2 was determined via quantitative PCR with the real time cycler Rotor-Gene™ 3000. Using the comparative method of the operating software the change of the gene expression rate of the stimulated probes compared to the unstimulated control could be shown. The normalisation was performed against the reference gene YWHAZ (Tyrosine 3-monooxygenase/tryptophan 5-monooxygenase activation protein). The results are shown in FIG. 2.

### Example 3

### Determination of the Specific QC Activity in Conditioned Medium of THP-1 Cells

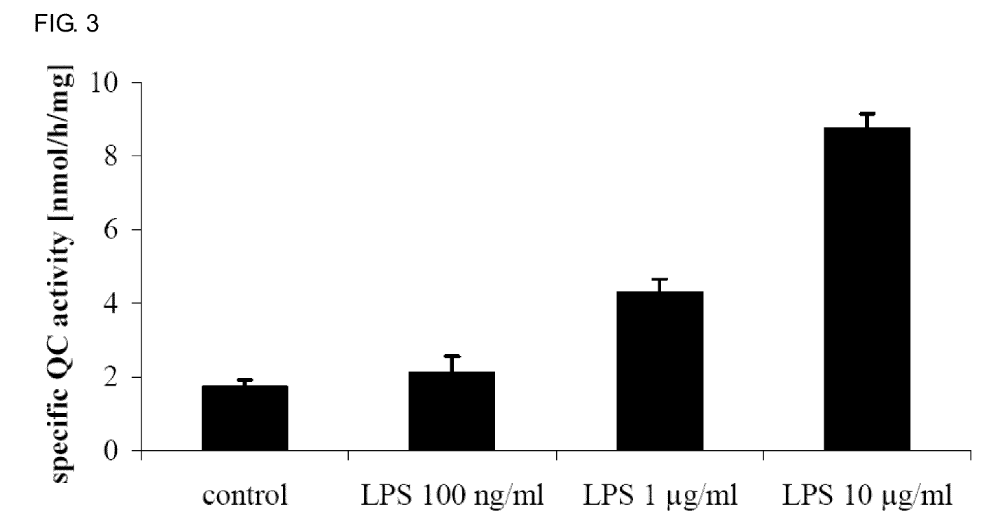

5×10⁶ THP-1 cells were seeded into 5 ml RPMI-1640 (Invitrogen) without phenol red and without FCS into 25 cm² suspension flasks (Greiner) and stimulated with different concentrations of LPS (Sigma). After 24 h incubation at 37° C. and 5% CO₂ cells were separated from the medium, which was reduced by centrifugation (4000×g) using U-Tube™ Concentrators 6-10 (Merck, Novagen) with a MWCO (Moleculare Weight Cut Off) 10 kDa to a final volume of 250 μl. The analysis of the protein concentration via Bradford method followed. The determination of the specific QC activity was realised by using a in-house established HPLC method. The results are shown in FIG. 3.

### Example 4

### Determination of QC Activity

Fluorometric Assays

All measurements were performed with a BioAssay Reader HTS-7000Plus for microplates (Perkin Elmer) at 30° C. QC activity was evaluated fluorometrically using H-Gln-bNA. The samples consisted of 0.2 mM fluorogenic substrate, 0.25 U pyroglutamyl aminopeptidase (Unizyme, Hørsholm, Denmark) in 0.2 M Tris/HCl, pH 8.0 containing 20 mM EDTA and an appropriately diluted aliquot of QC in a final volume of 250 μl. Excitation/emission wavelengths were 320/410 nm. The assay reactions were initiated by addition of glutaminyl cyclase. QC activity was determined from a standard curve of b-naphthylamine under assay conditions. One unit is defined as the amount of QC catalyzing the formation of 1 μmol pGlu-bNA from H-Gln-bNA per minute under the described conditions.

In a second fluorometric assay, QC activity was determined using H-Gln-AMC as substrate. Reactions were carried out at 30° C. utilizing the NOVOStar reader for microplates (BMG labtechnologies). The samples consisted of varying concentrations of the fluorogenic substrate, 0.1 U pyroglutamyl aminopeptidase (Qiagen) in 0.05 M Tris/HCl, pH 8.0 containing 5 mM EDTA and an appropriately diluted aliquot of QC in a final volume of 250 μl. Excitation/emission wavelengths were 380/460 nm. The assay reactions were initiated by addition of glutaminyl cyclase. QC activity was determined from a standard curve of 7-amino-4-methylcoumarin under assay conditions. The kinetic data were evaluated using GraFit software.

Spectrophotometric Assay of QC

In this assay, QC activity was analyzed spectrophotometrically using a continuous method, that was derived by adapting a previous discontinuous assay (Bateman, R. C. J. 1989 J Neurosci Methods 30, 23-28) utilizing glutamate dehydrogenase as auxiliary enzyme. Samples consisted of the respective QC substrate, 0.3 mM NADH, 14 mM a-Ketoglutaric acid and 30 U/ml glutamate dehydrogenase in a final volume of 250 μl. Reactions were started by addition of QC and persued by monitoring of the decrease in absorbance at 340 nm for 8-15 min.

The initial velocities were evaluated and the enzymatic activity was determined from a standard curve of ammonia under assay conditions. All samples were measured at 30° C., using either the SPECTRAFluor Plus or the Sunrise (both from TECAN) reader for microplates. Kinetic data was evaluated using GraFit software.

What is claimed is:

1. A method for diagnosing a neurodegenerative disease in a subject, the method comprising:
detecting an amount of glutaminyl cyclase (QC), or an isoform thereof, in a biological sample of said subject; and
comparing the detected amount of QC in the biological sample with an amount of QC characteristic of a normal control;
wherein
an elevated amount of QC in said biological sample relative to the normal control is a positive indicator of the neurodegenerative disease; and
the neurodegenerative disease is selected from the group consisting of Alzheimer's Disease (AD), Neurodegeneration in Down's Syndrome (NDS) and Mild Cognitive Impairment (MCI).

2. The method of claim 1 further comprising:
detecting an amount of Aβ N3pE-X,
comparing the detected amount Aβ N3pE-X in the biological sample with an amount of Aβ N3pE-X characteristic of a normal control;
wherein
an elevated amount of QC and Aβ N3pE-X in said biological sample relative to the normal control is a positive indicator of the neurodegenerative disease; and
X is an integer selected from the group consisting of 38, 40 and 42.

3. The method of claim 1 further comprising:
detecting an amount of a chemokine;
comparing the detected amount of the chemokine in the biological sample with an amount of chemokine characteristic of a normal control;
wherein an elevated amount of QC and chemokine in said biological sample relative to the normal control is a positive indicator of the neurodegenerative disease.

4. The method according to claim 1, wherein said QC is human QC or an isoform thereof, having an amino acid sequence selected from the group consisting of SEQ ID NO 1; SEQ ID NO: 2; SEQ IDNO: 3; SEQ ID: NO: 4; and SEQ ID NO: 5.

5. The method according to claim 4, wherein said QC is human QC of SEQ ID NO: 1.

6. The method according to claim 2, wherein said QC is human QC or an isoform thereof, having an amino acid sequence selected from the group consisting of SEQ ID NO 1; SEQ ID NO: 2; SEQ IDNO: 3; SEQ ID: NO: 4; and SEQ ID NO: 5.

7. The method according to claim 6, wherein said QC is human QC of SEQ ID NO: 1.

8. The method according to claim 3, wherein said QC is human QC or an isoform thereof, having an amino acid sequence selected from the group consisting of SEQ ID NO 1; SEQ ID NO: 2; SEQ IDNO: 3; SEQ ID: NO: 4; and SEQ ID NO: 5.

9. The method according to claim 8, wherein said QC is human QC of SEQ ID NO: 1.

10. The method according to claim 1, wherein said biological sample is serum, plasma, urine or cerebrospinal fluid.

11. The method according to claim 10, wherein said biological sample is plasma.

12. The method according to claim 2, wherein said biological sample is serum, plasma, urine or cerebrospinal fluid.

13. The method according to claim 12, wherein said biological sample is plasma.

14. The method according to claim 3, wherein said biological sample is serum, plasma, urine or cerebrospinal fluid.

15. The method according to claim 14, wherein said biological sample is plasma.

16. The method according to claim 1, wherein the amount of QC is detected by immunoturbidimetric assay, immunofluorescence, immunodiffusion, enzyme-linked immunosorbent assay (ELISA), radioimmunoassay (RIA), Western blot, protein activity assay, Northern Blot, PCR, high performance liquid chromatography (HPLC), mass spectrometry (MS), gas chromatography (GC), GC-MS, LC-MS, or LC-MS/MS.

17. The method according to claim 2, wherein the amount of QC is detected by immunoturbidimetric assay, immunofluorescence, immunodiffusion, enzyme-linked immunosorbent assay (ELISA), radioimmunoassay (RIA), Western blot, protein activity assay, Northern Blot, PCR, high performance liquid chromatography (HPLC), mass spectrometry (MS), gas chromatography (GC), GC-MS, LC-MS, or LC-MS/MS.

18. The method according to claim 3, wherein the amount of QC is detected by immunoturbidimetric assay, immunofluorescence, immunodiffusion, enzyme-linked immunosorbent assay (ELISA), radioimmunoassay (RIA), Western blot, protein activity assay, Northern Blot, PCR, high performance liquid chromatography (HPLC), mass spectrometry (MS), gas chromatography (GC), GC-MS, LC-MS, or LC-MS/MS.

19. The method according to claim 1, wherein the amount of QC, or an isoform thereof, is detected on the basis of the protein level of said QC or isoform thereof.

20. The method according to claim 2, wherein the amount of QC, or an isoform thereof, is detected on the basis of the protein level of said QC or isoform thereof.

21. The method according to claim 3, wherein the amount of QC, or an isoform thereof, is detected on the basis of the protein level of said QC or isoform thereof.

22. The method according to claim 1, wherein the amount of QC is detected using an antibody that specifically binds to QC, or an isoform thereof.

23. The method according to claim 2, wherein the amount of QC is detected using an antibody that specifically binds to QC, or an isoform thereof.

24. The method according to claim 3, wherein the amount of QC is detected using an antibody that specifically binds to QC, or an isoform thereof.

25. The method according to claim 1, wherein the amount of QC is detected by measuring the enzymatic activity of QC, or an isoform thereof.

26. The method according to claim 2, wherein the amount of QC is detected by measuring the enzymatic activity of QC, or an isoform thereof.

27. The method according to claim 3, wherein the amount of QC is detected by measuring the enzymatic activity of QC, or an isoform thereof.

28. The method according to claim 1, wherein the amount of QC, or an isoform thereof, is detected on the basis of the mRNA level of said QC or isoform thereof.

29. The method according to claim 2, wherein the amount of QC, or an isoform thereof, is detected on the basis of the mRNA level of said QC or isoform thereof.

30. The method according to claim 3, wherein the amount of QC, or an isoform thereof, is detected on the basis of the mRNA level of said QC or isoform thereof.

31. The method of claim 2, wherein X is 42.

32. The method of claim 2, wherein X is 40.

33. The method of claim 2, wherein X is 38.

34. The method according to claim 2, wherein detecting an amount of Aβ N3pE-X comprises detecting (i) one or more of Aβ N3pE-42, Aβ N3pE-40, and Aβ N3pE-38 and (ii) at least one of pGluABri or pGluADan.

35. The method according to claim 34, wherein detecting an amount of Aβ N3pE-X comprises detecting (i) two or more of Aβ N3pE-42, Aβ N3pE-40, and Aβ N3pE-38 and (ii) at least one of pGluABri or pGluADan.

36. The method according to claim 3, wherein said chemokine is selected from CCL2, CCL7, CCL8, CCL9/10, CCL13, CCL15, CCL16, CCL25 and Fractalkine.

37. The method according to claim 36, wherein said chemokine is CCL2.

38. The method of claim 1 further comprising:
obtaining a biological sample from said subject;
wherein detecting the amount of glutaminyl cyclase (QC), or an isoform thereof, in the biological sample of said subject comprises
contacting said biological sample with an antibody that binds to glutaminyl cyclase (QC), or its isoforms;
allowing the antibody and QC to form an immune complex; and
detecting the amount of immune complex formed as an indication of the amount of QC in said biological sample.

39. The method of claim 1, wherein detecting the amount of QC, or an isoform thereof, occurs in vitro.

40. The method of claim 2, wherein detecting the amount of QC and Aβ N3pE-X occurs in vitro.

41. The method of claim 3, wherein detecting the amount of QC and chemokine occurs in vitro.

42. The method of claim 1, wherein the neurodegenerative disease is AD.

43. The method of claim 1, wherein the neurodegenerative disease is NDS.

44. The method of claim 1, wherein the neurodegenerative disease is MCI.

45. A kit for diagnosing a neurodegenerative disease comprising an antibody that binds to QC and an established standard of an amount of QC characteristic of a normal control.

46. The kit of claim 27, wherein the neurodegenerative disease is selected from the group consisting of Alzheimer's Disease (AD), Neurodegeneration in Down's Syndrome (NDS) and Mild Cognitive Impairment (MCI).
